(19) United States
(12) Patent Application Publication
(10) Pub. No.: US 2012/0135969 A1
(43) Pub. Date: May 31, 2012

(54) NOVEL POWDERED CRYSTALLINE MEDICINES FOR INHALATION

(75) Inventors: Claudius Weiler, Ingelheim-Grobwinternheim (DE); Marc Egen, Ingelheim (DE)

(73) Assignee: BOEHRINGER INGELHEIM INTERNATIONAL GMBH, Ingelheim am Rhein (DE)

(21) Appl. No.: 13/131,386

(22) PCT Filed: Nov. 23, 2009
(86) PCT No.: PCT/EP09/65621

§ 371 Date: Jun. 24, 2011

(30) Foreign Application Priority Data
Nov. 27, 2008 (EP) 08170072.6

(51) Int. Cl. A61K 47/10; B29B 9/12; A61P 11/06; A61K 31/56; A61P 11/00

(52) U.S. Cl. 514/169; 514/772; 264/12

(57) ABSTRACT

The invention relates to manufacturing processes for preparing inhalable powders, and to the stable crystalline inhalable powders prepared by this process. Similarly, the invention relates to the use of these inhalable powders for preparing a medicament for the treatment of respiratory complaints, particularly for the treatment of COPD (chronic obstructive pulmonary disease) and asthma.

The invention relates to stable medicament compositions for administration by inhalation, wherein one or more active substances are embedded in a crystalline matrix of an adjuvant. The invention relates particularly to spray-dried, crystalline and storage-stable powders, wherein one or more active substances are embedded in a crystalline mannitol matrix. Moreover, the invention relates to processes for preparing them and their use for preparing a medicament for the treatment of respiratory diseases, particularly for the treatment of COPD (chronic obstructive pulmonary disease) and asthma.

### BACKGROUND TO THE INVENTION

In the form of powders for inhalation, inhalable powders packed for example into suitable capsules (inhalettes) are delivered to the lungs by means of powder inhalers. Other systems are also known in which the quantity of powder to be administered is pre-dosed (e.g. blisters), and multi-dose powder systems are also known. Alternatively, the medicaments may be administered by inhalation of suitable powdered inhalable aerosols which are suspended for example in HFA134a, HFA227 or mixtures thereof, as propellant gas.

During powder inhalation, the microparticles of a pure active substance are administered through the airways to the surface of the lungs, e.g. in the alveoli, by the inhalation process. These particles are deposited on the surface and are only absorbed into the body after the dissolving process by active and passive transporting processes.

Inhalation systems are known in the literature in which the active substance is present in the form of solid particles, either as a micronised suspension in a suitable solvent system as carrier, or in the form of a dry powder.

Usually, powder inhalants, e.g. in the form of capsules for inhalation, are prepared on the basis of the general teaching, as described in DE-A-179 22 07.

A critical factor in multi-substance systems of this kind is the uniform distribution of the medicament in the powder mixture.

Another significant aspect with powder inhalants is that during the inhalative administration of the active substance only particles of a specific aerodynamic size reach the target organ, the lungs. The mean particle size of these lung-bound particles (inhalable fraction) is in the region of a few microns, typically between 0.1 and 10 μm, preferably below 6 μm. Particles of this kind are usually produced by micronisation (air jet milling). Frequently, this results in particles of this kind being of complex composition in terms of their crystalline properties due to this mechanical step.

It is known from the literature that particles in the region of less than 10 μm may be prepared by spray-drying. The spray-drying of pure active substances for inhalation purposes (powder inhalation) is also described in the prior art [e.g.: EP 0 072 046 A1; WO 2000 000176 A1; U.S. Pat. No. 6,019,968; A. Chawla, K. M. G. Taylor, J. M. Newton, M. C. R. Johnson, Int. J. Pharm, 108 (3), (1994), 233-240].

Particularly in the field of lung therapy, spray-drying is a suitable method of preparing peptide/protein-containing powders for treating various ailments [U.S. Pat. No. 5,626,874; U.S. Pat. No. 5,972,388]

Formulation systems are known to the skilled man in which co-spray micronisates of active substances and physiologically acceptable excipients [WO 9952506] for inhalative use are disclosed. Also known are powder preparations containing co-spray micronisates of SLPI protein in physiologically acceptable carrier materials [WO 9917800]; co-spray-dried interferon with a carrier material [WO 9531479] and co-spray micronisates consisting of an active substance and cellulose derivatives [WO 9325198].

The specific use of mannitol as adjuvant for co-spray micronisates for stabilising peptides and proteins is described in WO 05/020953. Formulations are disclosed which are characterised in that complex proteins are present in an amorphous matrix in the form of embedding particles that are distinguished by their good long-term stability and inhalability.

Conventional inhalative spray-drying formulations and the methods of producing them are thus based on the concept that the amorphous or vitreous state primarily obtained by the spray-drying is stabilised and maintained for the purpose of long-term stability. The property of long-term stability in the sense of the invention can be simulated by stress storage. Storage under the conditions of “40° C. at 75% relative humidity, stored open for 1 week” can be used as a feature to arrive at an evaluation as to whether an inhalative formulation is classified as long-term stable. After-drying processes as disclosed for example in WO 05/020953 serve to reduce the water content in order to stabilise the amorphous state of these spray drying formulations, which may be accompanied by unregulated crystallisation.

The aim of the invention is to provide crystalline particles for inhalative use in which at least one pharmaceutically active substance is embedded in a crystalline matrix of an adjuvant.

A further aim of the invention is to make the stabilising activity of the crystalline state useful for spray-dried inhalable embedding particles.

A fundamental aim of the invention is to provide spray-dried powders which are characterised by good long-term stability and inhalability. It is crucial to achieve a balance between the two criteria.

A further aim of the invention is to provide manufacturing methods for preparing the inhalable powders according to the invention.

A further aim of the invention is to provide pharmaceutical preparations for inhalative use, be it in the form of a dry powder, a propellant gas-containing metered dose aerosol or a propellant gas-free inhalable solution.

### SUMMARY OF THE INVENTION

Surprisingly it has been found that the processes known from the prior art are not suitable for preparing inhalable powders that contain one or more pharmaceutical active substances and a matrix forming agent, the matrix forming agent being in crystalline form. The powders are characterised by a high degree of stability.

Within the scope of the present inventions, the term stable inhalable powders refers to those inhalable powders whose properties remain unchanged even over fairly long periods of time. Inhalable powders do not change their properties when there is both chemical stability of the individual components in the powder mixture and the physical or physicochemical stability thereof. This also presupposes that the components of the powder mixture remain unchanged in terms of their polymorphic and morphological properties. For inhalable powders the physical stability is of crucial importance.

In a preferred embodiment the powder consists predominantly of finely divided inhalable particles with a mean aerodynamic particle size (mass median aerodynamic diameter=MMAD) of ≦10 μm, preferably 0.5-7.5 μm, more preferably 1-5 μm. The matrix forming agents may be sugars, polyols, polymers or a combination of these. Polyols are preferred, while mannitol has an outstanding role.

Powders according to the invention are characterised in that they have a large inhalable fraction. The Fine Particle Dose represents the quantity of inhalable active substance particles (<5 μm), as may be determined on the basis of Pharm. Eur. 2.9.18 (European Pharmacopoeia, 6th edition 2008, Apparatus D—Andersen Cascade Impactor) or USP30-NF25 <601>.

Within the scope of the present invention the inhalable particles are determined as the “proportion by volume<5 μm after delivery”. By this is meant the proportion of the inhalable powder comprising particles smaller than 5 μm, measured by laser diffraction (given in [%]). The aerosol mist is produced by breaking up the sample by expelling it from an inhaler (Handihaler).

The powders according to the invention are formulations of pharmaceutical products, predominantly for administration by inhalation, which contain on of the powders according to the invention described here. In this context the invention also encompasses pharmaceutical compositions which contain the powders according to the invention as propellant-containing metered dose aerosols or as propellant-free inhalable solutions.

The invention further provides a process consisting of spray-drying and an additional integrated second drying zone, for manufacturing preparations of crystalline spray-drying particles. Using this second drying zone first of all the solvent is eliminated and then the matrix-forming agent is crystallised in the spray tower. A second drying stage causes the crystallised particles to dry out completely even before being precipitated in the collecting container of the spray dryer.

The temperature for the spray-drying process is below 135° C. (inflow temperature), preferably below 105° C. for drying gas 1. For the second drying step ambient air is sucked in and fed into the process (drying gas 2), while the temperature is between 300° C. and 400° C. and the ratio of drying gas 1 to drying gas 2 is between 20 to 1 and 3 to 1. The resulting exit temperature is in the range from approx. 50° C. to 70° C.

The present invention provides spray-dried crystalline powders that have improved properties in terms of properties such as flowability, dispersibility and stability during storage and processing. The present invention is characterised by a high consistency of delivery of the active substance at varying flow rates. The present invention thus solves problems that have arisen with the previous developments of formulations, particularly when using spray-drying powders containing mannitol, as their insufficient crystallinity had a negative effect on the physical and chemical stability of the powders.

### DETAILED DESCRIPTION OF THE INVENTION

Using the process according to the invention for preparing an inhalable powder for pulmonary (or nasal) inhalation, the active substance (or a physiologically acceptable salt thereof) is incorporated as a solid in physically stable form in a crystalline solid matrix of an adjuvant.

By a suitable choice of adjuvants, using the process according to the invention the active substance can be incorporated in the solid matrix such that this adjuvant acts as a matrix-forming agent and thus improves the physical stability of the spray-dried particles.

Surprisingly it has been found that the crystalline matrix particles prepared by the process according to the invention solve the problems stated hereinbefore.

Matrix-forming agents may be in principle sugars, polyols, polymers, amino acids, di-, tri-, oligo-, polypeptides, proteins or salts. Examples of particularly suitable sugars that may be mentioned are raffinose and galactose. Examples of particularly suitable sugar alcohols that may be mentioned are mannitol, xylitol, maltitol, galactitol, arabinitol, adonitol, lactitol, sorbitolol (glucitol), pyranosylsorbitol, inositol, myoinositol and meso-erythritol, of which mannitol, xylitol, maltitol and sorbitolol are preferred. Examples of particularly suitable amino acids that may be mentioned are leucine, lysine and glycine, preferably leucine. Preferably, according to the invention, sugars and the corresponding alcohols thereof which have a Tg value of less than 40° C. have proved advantageous as matrix-forming agents. Mannitol has an outstanding part to play in this. The Tg of a powder can be determined experimentally by DSC (DSC=Differential Scanning Calorimetry) (Breen et al., 2001, Pharm. Res., 18(9), 1345-1353). The increase in heat capacity as a function of temperature is recorded.

A solid is referred to as crystalline if its smallest parts are arranged regularly. The opposite of this is amorphous. Methods of determining crystallinity are DSC, density measurement, X-ray diffraction, IR spectroscopy or NMR. By crystalline is meant, for the purposes of the present invention, that the powdered formulations have a crystallinity of at least 90%, preferably at least 92.5% and particularly at least 95%. Also particularly preferred are a crystallinity of at least 96%, at least 97%, at least 98%, and at least 99%. The crystallinity in the sense of the present invention can be determined according to the information provided in the section headed “Methods”.

Inhalable powders that are prepared by the manufacturing process according to the invention contain a pharmaceutical active substance. In an independent embodiment the inhalable powders, which are prepared using the manufacturing process according to the invention, contain a combination of 2 or 3 pharmaceutical active substances. By a “pharmaceutical active substance” is meant a substance, a medicament, a composition or a combination thereof, that has a pharmacological, generally beneficial, effect on an organism, an organ, and/or a cell, if the active substance is brought into contact with the organism, organ or cell. When administered to a patient, the effect may be local or systemic.

The chemical compounds listed hereinafter (active substances) may be used on their own or in combination as the medicament-relevant component of the inhalable powders according to the invention.

In the compounds mentioned below, W is a pharmacologically active substance and is selected (for example) from among the betamimetics, anticholinergics, corticosteroids, PDE4-inhibitors, LTD4-antagonists, EGFR-inhibitors, dopamine agonists, H1-antihistamines, PAF-antagonists and PI3-kinase inhibitors. Moreover, double or triple combinations of W may be combined and used in the device according to the invention. Combinations of W might be, for example:
W denotes a betamimetic, combined with an anticholinergic, corticosteroid, PDE4-inhibitor, EGFR-inhibitor or LTD4-antagonist,
W denotes an anticholinergic, combined with a betamimetic, corticosteroid, PDE4-inhibitor, EGFR-inhibitor or LTD4-antagonist,
W denotes a corticosteroid, combined with a PDE4-inhibitor, EGFR-inhibitor or LTD4-antagonist
W denotes a PDE4-inhibitor, combined with an EGFR-inhibitor or LTD4-antagonist
W denotes an EGFR-inhibitor, combined with an LTD4-antagonist.

The compounds used as betamimetics are preferably compounds selected from among albuterol, arformoterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmefamol, salmeterol, soterenol, sulphonterol, terbutaline, tiaramide, tolubuterol, zinterol, CHF-1035, HOKU-81, KUL-1248 and
3-(4-{6-[2-hydroxy-2-(4-hydroxy-3-hydroxymethyl-phenyl)-ethylamino]-hexyloxy}-butyl)-benzyl-sulphonamide
5-[2-(5,6-diethyl-indan-2-ylamino)-1-hydroxy-ethyl]-8-hydroxy-1H-quinolin-2-one
4-hydroxy-7-[2-{[2-{[3-(2-phenylethoxy)propyl]sulphonyl}ethyl]-amino}ethyl]-2(3H)-benzothiazolone
1-(2-fluoro-4-hydroxyphenyl)-2-[4-(1-benzimidazolyl)-2-methyl-2-butylamino]ethanol
1-[3-(4-methoxybenzyl-amino)-4-hydroxyphenyl]-2-[4-(1-benzimidazolyl)-2-methyl-2-butylamino]ethanol
1-[2H-5-hydroxy-3-oxo-4H-1,4-benzoxazin-8-yl]-2-[3-(4-N,N-dimethylaminophenyl)-2-methyl-2-propylamino]ethanol
1-[2H-5-hydroxy-3-oxo-4H-1,4-benzoxazin-8-yl]-2-[3-(4-methoxyphenyl)-2-methyl-2-propylamino]ethanol
1-[2H-5-hydroxy-3-oxo-4H-1,4-benzoxazin-8-yl]-2-[3-(4-n-butyloxyphenyl)-2-methyl-2-propylamino]ethanol
1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol
5-hydroxy-8-(1-hydroxy-2-isopropylaminobutyl)-2H-1,4-benzoxazin-3-(4H)-one
1-(4-amino-3-chloro-5-trifluoromethylphenyl)-2-tert.-butylamino)ethanol
6-hydroxy-8-{1-hydroxy-2-[2-(4-methoxy-phenyl)-1,1-dimethyl-ethylamino]-ethyl}-4H-benzo[1,4]oxazin-3-one
6-hydroxy-8-{1-hydroxy-2-[2-(ethyl 4-phenoxy-acetate)-1,1-dimethyl-ethylamino]-ethyl}-4H-benzo[1,4]oxazin-3-one
6-hydroxy-8-{1-hydroxy-2-[2-(4-phenoxy-acetic acid)-1,1-dimethyl-ethylamino]-ethyl}-4H-benzo[1,4]oxazin-3-one
8-{2-[1,1-dimethyl-2-(2,4,6-trimethylphenyl)-ethylamino]-1-hydroxy-ethyl}-6-hydroxy-4H-benzo[1,4]oxazin-3-one
6-hydroxy-8-{1-hydroxy-2-[2-(4-hydroxy-phenyl)-1,1-dimethyl-ethylamino]-ethyl}-4H-benzo[1,4]oxazin-3-one
6-hydroxy-8-{1-hydroxy-2-[2-(4-isopropyl-phenyl)-1,1dimethyl-ethylamino]-ethyl}-4H-benzo[1,4]oxazin-3-one
8-{2-[2-(4-ethyl-phenyl)-1,1-dimethyl-ethylamino]-1-hydroxy-ethyl}-6-hydroxy-4H-benzo[1,4]oxazin-3-one
8-{2-[2-(4-ethoxy-phenyl)-1,1-dimethyl-ethylamino]-1-hydroxy-ethyl}-6-hydroxy-4H-benzo[1,4]oxazin-3-one
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid
8-{2-[2-(3,4-difluoro-phenyl)-1,1-dimethyl-ethylamino]-1-hydroxy-ethyl}-6-hydroxy-4H-benzo[1,4]oxazin-3-one
1-(4-ethoxy-carbonylamino-3-cyano-5-fluorophenyl)-2-(tert-butylamino)ethanol
2-hydroxy-5-(1-hydroxy-2-{2-[4-(2-hydroxy-2-phenyl-ethylamino)-phenyl]-ethylamino}-ethyl)-benzaldehyde
N-[2-hydroxy-5-(1-hydroxy-2-{2-[4-(2-hydroxy-2-phenyl-ethylamino)-phenyl]-ethylamino}-ethyl)-phenyl]-formamide
8-hydroxy-5-(1-hydroxy-2-{2-[4-(6-methoxy-biphenyl-3-ylamino)-phenyl]-ethylamino}-ethyl)-1H-quinolin-2-one
8-hydroxy-5-[1-hydroxy-2-(6-phenethylamino-hexylamino)-ethyl]-1H-quinolin-2-one
5-[2-(2-{4-[4-(2-amino-2-methyl-propoxy)-phenylamino]-phenyl}-ethylamino)-1-hydroxy-ethyl]-8-hydroxy-1H-quinolin-2-one
[3-(4-{6-[2-hydroxy-2-(4-hydroxy-3-hydroxymethyl-phenyl)-ethylamino]-hexyloxy}-butyl)-5-methyl-phenyl]-urea
4-(2-{6-[2-(2,6-dichloro-benzyloxy)-ethoxy]-hexylamino}-1-hydroxy-ethyl)-2-hydroxymethyl-phenol
3-(4-{6-[2-hydroxy-2-(4-hydroxy-3-hydroxymethyl-phenyl)-ethylamino]-hexyloxy}-butyl)-benzylsulphonamide
3-(3-{7-[2-hydroxy-2-(4-hydroxy-3-hydroxymethyl-phenyl)-ethylamino]-heptyloxy}-propyl)-benzylsulphonamide
4-(2-{6-[4-(3-cyclopentanesulphonyl-phenyl)-butoxy]-hexylamino}-1-hydroxy-ethyl)-2-hydroxymethyl-phenol
N-adamantan-2-yl-2-(3-{2-[2-hydroxy-2-(4-hydroxy-3-hydroxymethyl-phenyl)-ethylamino]-propyl}-phenyl)-acetamide

optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The anticholinergics used are preferably compounds selected from among the tiotropium salts, preferably the bromide salt, oxitropium salts, preferably the bromide salt, flutropium salts, preferably the bromide salt, ipratropium salts, preferably the bromide salt, glycopyrronium salts, preferably the bromide salt, trospium salts, preferably the chloride salt, tolterodine. In the above-mentioned salts the cations are the pharmacologically active constituents. As anions the above-mentioned salts may preferably contain the chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred.

Other preferred anticholinergics are selected from among the salts of formula AC-1

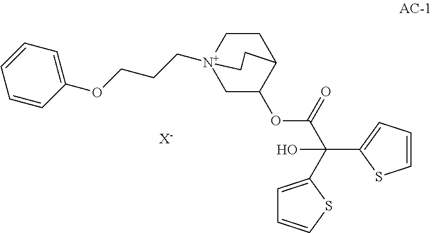

wherein X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate, preferably an anion with a single negative charge, particularly preferably an anion selected from among the fluoride, chloride, bromide, methanesulphonate and p-toluenesulphonate, particularly preferably bromide, optionally in the form of the racemates, enantiomers or hydrates thereof. Of particular importance are those pharmaceutical combinations which contain the enantiomers of formula AC-1-ene

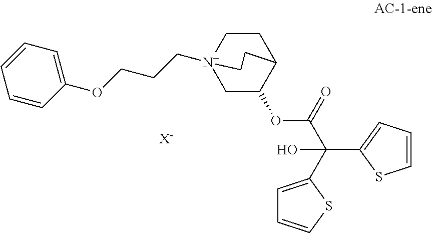

wherein X⁻ may have the above-mentioned meanings. Other preferred anticholinergics are selected from the salts of formula AC-2

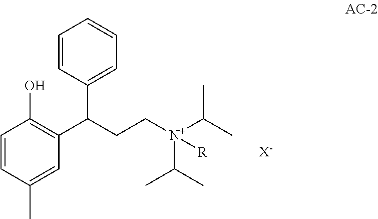

wherein R denotes either methyl or ethyl and wherein X⁻ may have the above-mentioned meanings. In an alternative embodiment the compound of formula AC-2 may also be present in the form of the free base AC-2-base.

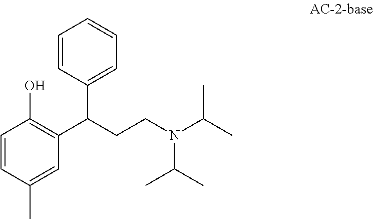

Other specified compounds are:
tropenol 2,2-diphenylpropionate methobromide
scopine 2,2-diphenylpropionate methobromide
scopine 2-fluoro-2,2-diphenylacetate methobromide
tropenol 2-fluoro-2,2-diphenylacetate methobromide
tropenol 3,3′,4,4′-tetrafluorobenzilate methobromide
scopine 3,3′,4,4′-tetrafluorobenzilate methobromide
tropenol 4,4′-difluorobenzilate methobromide
scopine 4,4′-difluorobenzilate methobromide
tropenol 3,3′-difluorobenzilate methobromide
scopine 3,3′-difluorobenzilate methobromide
tropenol 9-hydroxy-fluorene-9-carboxylate methobromide
tropenol 9-fluoro-fluorene-9-carboxylate methobromide
scopine 9-hydroxy-fluorene-9-carboxylate methobromide
scopine 9-fluoro-fluorene-9-carboxylate methobromide
tropenol 9-methyl-fluorene-9-carboxylate methobromide
scopine 9-methyl-fluorene-9-carboxylate methobromide
cyclopropyltropine benzilate methobromide;
cyclopropyltropine 2,2-diphenylpropionate methobromide
cyclopropyltropine 9-hydroxy-xanthene-9-carboxylate methobromide
cyclopropyltropine 9-methyl-fluorene-9-carboxylate methobromide
cyclopropyltropine 9-methyl-xanthene-9-carboxylate methobromide
cyclopropyltropine 9-hydroxy-fluorene-9-carboxylate methobromide
cyclopropyltropine methyl 4,4′-difluorobenzilate methobromide
tropenol 9-hydroxy-xanthene-9-carboxylate methobromide
scopine 9-hydroxy-xanthene-9-carboxylate methobromide
tropenol 9-methyl-xanthene-9-carboxylate methobromide
scopine 9-methyl-xanthene-9-carboxylate methobromide
tropenol 9-ethyl-xanthene-9-carboxylate methobromide
tropenol 9-difluoromethyl-xanthene-9-carboxylate methobromide
scopine 9-hydroxymethyl-xanthene-9-carboxylate methobromide

The above-mentioned compounds may also be used as salts within the scope of the present invention, wherein instead of the methobromide the salts metho-X are used, wherein X may have the meanings given hereinbefore for X⁻.

As corticosteroids it is preferable to use compounds selected from among beclomethasone, betamethasone, budesonide, butixocort, ciclesonide, deflazacort, dexamethasone, etiprednol, flunisolide, fluticasone, loteprednol, mometasone, prednisolone, prednisone, rofleponide, triamcinolone, RPR-106541, NS-126, ST-26 and
(S)-fluoromethyl 6,9-difluoro-17-[(2-furanylcarbonyl)oxy]-11-hydroxy-16-methyl-3-oxo-androsta-1,4-diene-17-carbothionate
(S)-(2-oxo-tetrahydro-furan-3S-yl) 6,9-difluoro-11-hydroxy-16-methyl-3-oxo-17-propionyloxy-androsta-1,4-diene-17-carbothionate,
cyanomethyl 6α,9α-difluoro-11β-hydroxy-16α-methyl-3-oxo-17α-(2,2,3,3-tertamethylcyclopropylcarbonyl)oxy-androsta-1,4-diene-17β-carboxylate

optionally in the form of the racemates, enantiomers or diastereomers thereof and optionally in the form of the salts and derivatives thereof, the solvates and/or hydrates thereof. Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, dichloroacetates, propionates, dihydrogen phosphates, palmitates, pivalates or furoates.

PDE4-inhibitors which may be used are preferably compounds selected from among enprofyllin, theophyllin, roflumilast, ariflo (cilomilast), tofimilast, pumafentrin, lirimilast, arofyllin, atizoram, D-4418, Bay-198004, BY343, CP-325.366, D-4396 (Sch-351591), AWD-12-281 (GW-842470), NCS-613, CDP-840, D-4418, PD-168787, T-440, T-2585, V-11294A, CI-1018, CDC-801, CDC-3052, D-22888, YM-58997, Z-15370 and
N-(3,5-dichloro-1-oxo-pyridin-4-yl)-4-difluoromethoxy-3-cyclopropylmethoxybenzamide
(−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide
(R)-(+)-1-(4-bromobenzyl)-4-[(3-cyclopentyloxy)-4-methoxyphenyl]-2-pyrrolidone
3-(cyclopentyloxy-4-methoxyphenyl)-1-(4-N′-[N-2-cyano-S-methyl-isothioureido]benzyl)-2-pyrrolidone
cis[4-cyano-4-(3-cyclopentyloxy-4-methoxyphenyl)cyclohexane-1-carboxylic acid]
2-carbomethoxy-4-cyano-4-(3-cyclopropylmethoxy-4-difluoromethoxy-phenyl)cyclohexan-1-one
cis[4-cyano-4-(3-cyclopropylmethoxy-4-difluoromethoxyphenyl)cyclohexan-1-ol]
(R)-(+)-ethyl[4-(3-cyclopentyloxy-4-methoxyphenyl)pyrrolidin-2-ylidene]acetate
(S)-(−)-ethyl[4-(3-cyclopentyloxy-4-methoxyphenyl)pyrrolidin-2-ylidene]acetate
9-cyclopentyl-5,6-dihydro-7-ethyl-3-(2-thienyl)-9H-pyrazolo[3,4-c]-1,2,4-triazolo[4,3-a]pyridine
9-cyclopentyl-5,6-dihydro-7-ethyl-3-(tert-butyl)-9H-pyrazolo[3,4-c]-1,2,4-triazolo[4,3-a]pyridine

optionally in the form of the racemates, enantiomers or diastereomers thereof and optionally in the form of the pharmacologically acceptable acid addition salts thereof, the solvates and/or hydrates thereof. According to the invention the acid addition salts of the PDE4 inhibitors are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The LTD4-antagonists used are preferably compounds selected from among montelukast, pranlukast, zafirlukast, MCC-847 (ZD-3523), MN-001, MEN-91507 (LM-1507), VUF-5078, VUF-K-8707, L-733321 and
1-(((R)-(3-(2-(6,7-difluoro-2-quinolinyl)ethenyl)phenyl)-3-(2-(2-hydroxy-2-propyl)phenyl)thio)methylcyclopropane-acetic acid,
1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid
[2-[[2-(4-tert-butyl-2-thiazolyl)-5-benzofuranyl]oxymethyl]phenyl]acetic acid

optionally in the form of the racemates, enantiomers or diastereomers thereof and optionally in the form of the pharmacologically acceptable acid addition salts, solvates and/or hydrates thereof. According to the invention these acid addition salts are preferably selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate. By salts or derivatives which the LTD4-antagonists may optionally be capable of forming are meant, for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

EGFR-inhibitors which may be used are preferably compounds selected from among cetuximab, trastuzumab, ABX-EGF, Mab ICR-62 and
4-[(3-chloro-4-fluorophenyl)amino]-6-{[4-(morpholin-4-yl)-1-oxo-2-buten-1-yl]amino}-7-cyclopropylmethoxy-quinazoline
4-[(3-chloro-4-fluorophenyl)amino]-6-{[4-(N,N-diethylamino)-1-oxo-2-buten-1-yl]-amino}-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopropylmethoxy-quinazoline
4-[(R)-(1-phenyl-ethyl)amino]-6-{[4-(morpholin-4-yl)-1-oxo-2-buten-1-yl]amino}-7-cyclopentyloxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline
4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline
4-[(3-chloro-4-fluoro-phenyl)amino]-6-[2-((S)-6-methyl-2-oxo-morpholin-4-yl)-ethoxy]-7-methoxy-quinazoline
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopentyloxy-quinazoline
4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline
4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline
4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline
4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline
4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline
4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline
4-[(3-chloro-4-fluorophenyl)amino]-6-{[4-(N-cyclopropyl-N-methyl-amino)-1-oxo-2-buten-1-yl]amino}-7-cyclopentyloxy-quinazoline
4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline
4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline
4-[(3-ethynyl-phenyl)amino]-6,7-bis-(2-methoxy-ethoxy)-quinazoline
4-[(3-chloro-4-fluorophenyl)amino]-7-[3-(morpholin-4-yl)-propyloxy]-6-[(vinyl-carbonyl)amino]-quinazoline
4-[(R)-(1-phenyl-ethyl)amino]-6-(4-hydroxy-phenyl)-7H-pyrrolo[2,3-d]pyrimidine
3-cyano-4-[(3-chloro-4-fluorophenyl)amino]-6-{[4-(N,N-dimethylamino)-1-oxo-2-buten-1-yl]amino}-7-ethoxy-quinoline
4-{[3-chloro-4-(3-fluoro-benzyloxy)-phenyl]amino}-6-(5-{[(2-methanesulphonyl-ethyl)amino]methyl}-furan-2-yl)quinazoline
4-[(R)-(1-phenyl-ethyl)amino]-6-{[4-((R)-6-methyl-2-oxo-morpholin-4-yl)-1-oxo-2-buten-1-yl]amino}-7-methoxy-quinazoline
4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline
4-[(3-chloro-4-fluorophenyl)amino]-6-({4-[N,N-to-(2-methoxy-ethyl)-amino]-1-oxo-2-buten-1-yl}amino)-7-[(tetrahydrofuran-2-yl)methoxy]-quinazoline
4-[(3-ethynyl-phenyl)amino]-6-{[4-(5,5-dimethyl-2-oxo-morpholin-4-yl)-1-oxo-2-buten-1-yl]amino}-quinazoline
4-[(3-chloro-4-fluoro-phenyl)amino]-6-[2-(2,2-dimethyl-6-oxo-morpholin-4-yl)-ethoxy]-7-methoxy-quinazoline
4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline
4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline
4-[(3-chloro-4-fluoro-phenyl)amino]-6-{2-[4-(2-oxo-morpholin-4-yl)-piperidin-1-yl]-ethoxy}-7-methoxy-quinazoline
4-[(3-chloro-4-fluoro-phenyl)amino]-6-[1-(tert.-butyloxycarbonyl)-piperidin-4-yloxy]-7-methoxy-quinazoline
4-[(3-chloro-4-fluoro-phenyl)amino]-6-(trans-4-amino-cyclohexan-1-yloxy)-7-methoxy-quinazoline
4-[(3-chloro-4-fluoro-phenyl)amino]-6-(trans-4-methanesulphonylamino-cyclohexan-1-yloxy)-7-methoxy-quinazoline
4-[(3-chloro-4-fluoro-phenyl)amino]-6-(tetrahydropyran-3-yloxy)-7-methoxy-quinazoline
4-[(3-chloro-4-fluoro-phenyl)amino]-6-(1-methyl-piperidin-4-yloxy)-7-methoxy-quinazoline
4-[(3-chloro-4-fluoro-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[(methoxymethyl)carbonyl]-piperidin-4-yloxy}-7-methoxy-quinazoline
4-[(3-chloro-4-fluoro-phenyl)amino]-6-(piperidin-3-yloxy)-7-methoxy-quinazoline
4-[(3-chloro-4-fluoro-phenyl)amino]-6-[1-(2-acetylamino-ethyl)-piperidin-4-yloxy]-7-methoxy-quinazoline
4-[(3-chloro-4-fluoro-phenyl)amino]-6-(tetrahydropyran-4-yloxy)-7-ethoxy-quinazoline
4-[(3-chloro-4-fluoro-phenyl)amino]-6-((S)-tetrahydrofuran-3-yloxy)-7-hydroxy-quinazoline
4-[(3-chloro-4-fluoro-phenyl)amino]-6-(tetrahydropyran-4-yloxy)-7-(2-methoxy-ethoxy)-quinazoline
4-[(3-chloro-4-fluoro-phenyl)amino]-6-{trans-4-[(dimethylamino)sulphonylamino]-cyclohexan-1-yloxy}-7-methoxy-quinazoline
4-[(3-chloro-4-fluoro-phenyl)amino]-6-{trans-4-[(morpholin-4-yl)carbonylamino]-cyclohexan-1-yloxy}-7-methoxy-quinazoline
4-[(3-chloro-4-fluoro-phenyl)amino]-6-{trans-4-[(morpholin-4-yl)sulphonylamino]-cyclohexan-1-yloxy}-7-methoxy-quinazoline
4-[(3-chloro-4-fluoro-phenyl)amino]-6-(tetrahydropyran-4-yloxy)-7-(2-acetylamino-ethoxy)-quinazoline
4-[(3-chloro-4-fluoro-phenyl)amino]-6-(tetrahydropyran-4-yloxy)-7-(2-methanesulphonylamino-ethoxy)-quinazoline
4-[(3-chloro-4-fluoro-phenyl)amino]-6-{1-[(piperidin-1-yl)carbonyl]-piperidin-4-yloxy}-7-methoxy-quinazoline
4-[(3-chloro-4-fluoro-phenyl)amino]-6-(1-aminocarbonylmethyl-piperidin-4-yloxy)-7-methoxy-quinazoline
4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline
4-[(3-chloro-4-fluoro-phenyl)amino]-6-(trans-4-ethanesulphonylamino-cyclohexan-1-yloxy)-7-methoxy-quinazoline
4-[(3-chloro-4-fluoro-phenyl)amino]-6-(1-methanesulphonyl-piperidin-4-yloxy)-7-ethoxy-quinazoline
4-[(3-chloro-4-fluoro-phenyl)amino]-6-(1-methanesulphonyl-piperidin-4-yloxy)-7-(2-methoxy-ethoxy)-quinazoline
4-[(3-chloro-4-fluoro-phenyl)amino]-6-[1-(2-methoxy-acetyl)-piperidin-4-yloxy]-7-(2-methoxy-ethoxy)-quinazoline
4-[(3-chloro-4-fluoro-phenyl)amino]-6-(cis-4-acetylamino-cyclohexan-1-yloxy)-7-methoxy-quinazoline
4-[(3-ethynyl-phenyl)amino]-6-[1-(tert.-butyloxycarbonyl)-piperidin-4-yloxy]-7-methoxy-quinazoline
4-[(3-ethynyl-phenyl)amino]-6-(tetrahydropyran-4-yloxy]-7-methoxy-quinazoline
4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline
4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline
4-[(3-chloro-4-fluoro-phenyl)amino]-6-{cis-4-[(morpholin-4-yl)carbonylamino]-cyclohexan-1-yloxy}-7-methoxy-quinazoline
4-[(3-chloro-4-fluoro-phenyl)amino]-6-{1-[2-(2-oxopyrrolidin-1-yl)ethyl]-piperidin-4-yloxy}-7-methoxy-quinazoline
4-[(3-chloro-4-fluoro-phenyl)amino]-6-{1-[(morpholin-4-yl)carbonyl]-piperidin-4-yloxy}-7-(2-methoxy-ethoxy)-quinazoline
4-[(3-ethynyl-phenyl)amino]-6-(1-acetyl-piperidin-4-yloxy)-7-methoxy-quinazoline
4-[(3-ethynyl-phenyl)amino]-6-(1-methyl-piperidin-4-yloxy)-7-methoxy-quinazoline
4-[(3-ethynyl-phenyl)amino]-6-(1-methanesulphonyl-piperidin-4-yloxy)-7-methoxy-quinazoline
4-[(3-chloro-4-fluoro-phenyl)amino]-6-(1-methyl-piperidin-4-yloxy)-7(2-methoxy-ethoxy)-quinazoline
4-[(3-chloro-4-fluoro-phenyl)amino]-6-(1-isopropyloxycarbonyl-piperidin-4-yloxy)-7-methoxy-quinazoline
4-[(3-chloro-4-fluoro-phenyl)amino]-6-(cis-4-methylamino-cyclohexan-1-yloxy)-7-methoxy-quinazoline
4-[(3-chloro-4-fluoro-phenyl)amino]-6-{cis-4-[N-(2-methoxy-acetyl)-N-methyl-amino]-cyclohexan-1-yloxy}-7-methoxy-quinazoline
4-[(3-ethynyl-phenyl)amino]-6-(piperidin-4-yloxy)-7-methoxy-quinazoline
4-[(3-ethynyl-phenyl)amino]-6-[1-(2-methoxy-acetyl)-piperidin-4-yloxy]-7-methoxy-quinazoline
4-[(3-ethynyl-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[(cis-2,6-dimethyl-morpholin-4-yl)carbonyl]-piperidin-4-yloxy}-7-methoxy-quinazoline
4-[(3-chloro-4-fluoro-phenyl)amino]-6-{1-[(2-methyl-morpholin-4-yl)carbonyl]-piperidin-4-yloxy}-7-methoxy-quinazoline
4-[(3-chloro-4-fluoro-phenyl)amino]-6-{1-[(S,S)-(2-oxa-5-aza-bicyclo[2.2.1]hept-5-yl)carbonyl]-piperidin-4-yloxy}-7-methoxy-quinazoline
4-[(3-chloro-4-fluoro-phenyl)amino]-6-{1-[(N-methyl-N-2-methoxyethyl-amino)carbonyl]-piperidin-4-yloxy}-7-methoxy-quinazoline
4-[(3-chloro-4-fluoro-phenyl)amino]-6-(1-ethyl-piperidin-4-yloxy)-7-methoxy-quinazoline
4-[(3-chloro-4-fluoro-phenyl)amino]-6-{1-[(2-methoxyethyl)carbonyl]-piperidin-4-yloxy}-7-methoxy-quinazoline
4-[(3-chloro-4-fluoro-phenyl)amino]-6-{1-[(3-methoxypropyl-amino)-carbonyl]-piperidin-4-yloxy}-7-methoxy-quinazoline
4-[(3-chloro-4-fluoro-phenyl)amino]-6-[cis-4-(N-methanesulphonyl-N-methyl-amino)-cyclohexan-1-yloxy]-7-methoxy-quinazoline
4-[(3-chloro-4-fluoro-phenyl)amino]-6-[cis-4-(N-acetyl-N-methyl-amino)-cyclohexan-1-yloxy]-7-methoxy-quinazoline
4-[(3-chloro-4-fluoro-phenyl)amino]-6-(trans-4-methylamino-cyclohexan-1-yloxy)-7-methoxy-quinazoline
4-[(3-chloro-4-fluoro-phenyl)amino]-6-[trans-4-(N-methanesulphonyl-N-methyl-amino)-cyclohexan-1-yloxy]-7-methoxy-quinazoline
4-[(3-chloro-4-fluoro-phenyl)amino]-6-(trans-4-dimethylamino-cyclohexan-1-yloxy)-7-methoxy-quinazoline
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline
4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline
4-[(3-chloro-4-fluoro-phenyl)amino]-6-(1-methanesulphonyl-piperidin-4-yloxy)-7-methoxy-quinazoline
4-[(3-chloro-4-fluoro-phenyl)amino]-6-(1-cyano-piperidin-4-yloxy)-7-methoxy-quinazoline

optionally in the form of the racemates, enantiomers, diastereomers thereof and optionally in the form of the pharmacologically acceptable acid addition salts, solvates or hydrates thereof. According to the invention these acid addition salts are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The dopamine agonists used are preferably compounds selected from among bromocriptine, cabergoline, alpha-dihydroergocryptine, lisuride, pergolide, pramipexole, roxindole, ropinirole, talipexole, terguride and viozan, optionally in the form of the racemates, enantiomers, diastereomers thereof and optionally in the form of the pharmacologically acceptable acid addition salts, solvates or hydrates thereof. According to the invention these acid addition salts are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate.

H1-Antihistamines which may be used are preferably compounds selected from among epinastine, cetirizine, azelastine, fexofenadine, levocabastine, loratadine, mizolastine, ketotifen, emedastine, dimetindene, clemastine, bamipine, cexchlorpheniramine, pheniramine, doxylamine, chlorphenoxamine, dimenhydrinate, diphenhydramine, promethazine, ebastine, desloratidine and meclozine, optionally in the form of the racemates, enantiomers, diastereomers thereof and optionally in the form of the pharmacologically acceptable acid addition salts, solvates or hydrates thereof. According to the invention these acid addition salts are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The pharmaceutically effective substances, formulations or mixtures of substances used may be any inhalable compounds, including also for example inhalable macromolecules, as disclosed in EP 1 003 478. Preferably, substances, formulations or mixtures of substances for treating respiratory complaints which are administered by inhalation are used.

In addition, the compound may come from the group of ergot alkaloid derivatives, the triptans, the CGRP-inhibitors, the phosphodiesterase-V inhibitors, optionally in the form of the racemates, enantiomers or diastereomers thereof, optionally in the form of the pharmacologically acceptable acid addition salts, the solvates and/or hydrates thereof.

Examples of ergot alkaloid derivatives are dihydroergotamine and ergotamine.

Generally, the proportion of the corresponding matrix forming agent in the powders according to the invention is more than 20% (w/w), particularly preferably more than 30% (w/w) of the dry mass of the powder. In another preferred embodiment of the invention the proportion of the corresponding matrix forming agent, e.g. the polyol or mannitol fraction is more than 20% (w/w) of the dry mass of the powder, preferably between 30-80% (w/w), particularly preferably between 30-70% (w/w). Corresponding to these embodiments, the proportion of the corresponding matrix forming agent will therefore be approximately 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% (w/w) of the dry mass of the powder. The corresponding embodiments apply particularly to powders in which a polyol, and especially mannitol, is used as matrix forming agent.

In another preferred embodiment the powder according to the invention contains matrix forming agents in a concentration such that the ratio of active substance : matrix forming agent is 1:999 to 1:1, particularly preferably 1:99 to 1:2 (units:w/w). According to these data the term active substance should also be understood as referring equally to a combination of active substances.

If the powders according to the invention contain anticholinergics, preferably combined with betamimetics and steroids, the proportion of the active substance or of the total active substances is normally between 0.1 and 50% (w/w), preferably between 0.2 and 40% (w/w), also preferably between 0.2 and 30% (w/w) and between 0.2 and 20% (w/w) of the total weight of the powder.

In another preferred form according to the invention the inhalable powders contain a pharmaceutical active substance selected from among the EGFR antagonists. Inhalable powders according to the invention comprising an active substance from this group of active substances have an active substance content which may be between 10 and 80% (w/w), preferably between 20 and 80% (w/w), particularly preferably between 30 and 80% (w/w) of the total weight of the powder.

Also preferred is an embodiment in which mannitol is exclusively used as the matrix forming agent.

The invention includes corresponding manufacturing processes for producing inhalable powders according to the invention. Such powders may be used both directly as powdered inhalants (multi-dose systems, pre-metered multi-dose systems and single-dose systems) and also as components that are mixed with additional (e.g. coarse-grained) adjuvant.

Within the scope of the present invention it has surprisingly been found that the efficient secondary drying of spray-dried powders which contain as matrix forming agent sugars, amino acids or polyols, preferably mannitol and leucine, particularly preferably mannitol, are particularly storage-stable, particularly at temperatures above 20° C., and are characterised by a high dispersibility, this secondary drying being carried out in the spray chamber by supplying a second drying gas. Thus, by supplying the second drying gas, a second drying step is carried out, which takes place within the spray drying process even before the particle deposition has occurred. The energy input of the second drying step should preferably be selected so that the exit temperature is in the range from 40° C. to 100° C.

Preparation processes according to the invention comprise the following steps:
(a) dissolving one or more active substances and the matrix forming agent in water, an organic solvent or an organic aqueous solvent mixture, in order to prepare a solution having a solids content of between 1% by weight and 20% by weight, preferably between 2% by weight and 10% by weight, particularly preferably between 3% by weight and 8% by weight,
(b) spraying the solution thus obtained in the conventional manner so as to obtain a spray mist with a droplet size having
   (i) a characteristic value Q_{(5.8)} of between 50% and 100% and
   (ii) an average droplet size X₅₀ in the range from 1 μm to 20 μm, preferably from 1 μm to 8 μm, particularly preferably from 1 μm to 3 μm,
(c) drying the spray mist thus obtained using a drying gas, with the application of the following parameters:
   (i) an entry temperature of the drying gas (1) of from 80° C. to 200° C., preferably from 80° C. to 150° C., more preferably from 90° C. to 160° C., more preferably from 90° C. to 140° C., more preferably from 100° C. to 150° C. and particularly preferably from 100° C. to 130° C.
   (ii) drying the aerosol in the spray chamber using a second drying gas (2), the temperature of the drying gas (2) being between 200° C. and 400° C.,
   (iii) the ratio of the volume flow of drying gas (1):drying gas (2) being between 20:1 and 3:1,
   (iv) the drying gas coefficient V1 being between 100 K and 2000 K and the drying coefficient V2 being between 250 K and 4000 K and
   (v) an exit temperature of the drying gas of from 40° C. to 90° C. and
(d) separating the dried particles of solid from the current of drying gas in conventional manner.

It has proved suitable to dissolve the active substance or a combination of active substances with one or more excipients, preferably with a polyol, particularly preferably with mannitol in water, an organic solvent or an organic-aqueous solvent mixture. The solvents used according to the invention, apart from water, are organic solvents with a boiling point of between 40° C. and 130° C., preferably alcohols. Preferably organic solvents are used which are either pharmaceutically acceptable or can be eliminated from the pharmaceutical formulation sufficiently (possibly to a standard required by the licensing authorities). Particularly preferably, ethanol, methanol, propanol, dichloromethane, water or a mixture of these solvents are used according to the invention.

The solids concentration of the spray solution serves to make the process economical. However, there are limits to be set on the concentration of active substances, as a result of the fact that the surface properties of the particles, including their particle size, can be optimised by a specific ratio between the droplet size and the solids concentration. Usually, a concentration of between 1% by weight and 20% by weight, preferably between 2% by weight and 10% by weight, most preferably between 3% by weight and 8% by weight is desirable. The droplet size that should be selected for the process can be characterised by the parameter X₅₀, which is in the range from 1 μm to 20 μm, preferably from 1 μm to 8 μm and particularly preferably from 1 μm to 3 μm, and the characteristic value Q_{(5.8)}, which is between 30% and 100% and preferably between 60% and 100%. The parameter X₅₀ for the droplet size represents the average, volume-related droplet size. The characteristic value Q_{(5.8)} denotes the particle size of the droplets, which is less than 5.8 pm based on the volume distribution of the droplets. The droplet sizes were determined within the scope of the present invention by laser diffraction (Fraunhofer diffraction). More detailed information on this can be found in the experimental descriptions of the invention.

This is converted into technical practicalities within the scope of the spray drying, using a corresponding commercial nozzle, e.g. a dual substance nozzle, which has these characteristics as a function of the nebulising pressure applied and the resulting mass flow of the nebulising gas as well as the spray rate (volume flow of the “spray solution”). Besides the special conditions that have to be adhered to in the actual spray process in order to generate suitable droplets for the drying process, it is apparent that the properties of the particles can be positively/deliberately influenced by the choice of drying parameters.

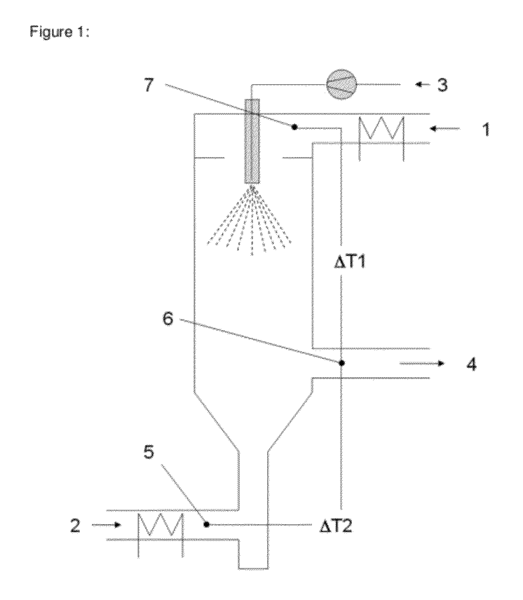

The process according to the invention is characterised in that the spray mist is subjected to a drying process by the introduction of at least two drying gas currents. It has proved advantageous if the drying gas current (1) is introduced into the spray chamber in the immediate vicinity of the production of the spray mist. By contrast, the introduction of the second drying gas as a supplementary secondary drying of the aerosol takes place even before the depositing of the particles by the influx of a drying gas (2) in the opposite direction of flow inside the spray chamber. This secondary drying step is also characterised in that the secondary drying of the spray-dried particles is carried out such that the particles are present in aerosol form, preferably in the spray chamber. The aerosol obtained by such a process, consisting of dried particles that are present in a dispersed state in the volume current of the drying gas is removed from the spray chamber (see FIG. 1: Exit from the spray chamber, designated by reference numeral 4). The dried solid particles are separated from the drying gas current by conventional methods. They may be separated off for example using a cyclone.

Characteristic values that impinge on the drying step are the entry temperature and mass flow of the drying gas (1) and of drying gas (2) and the mass flow of the spray liquid (MI) and the exit temperature of the drying gas. The ratio of the mass flow of the respective drying gas (Mg1, Mg2) and the mass flow of the spray liquid (MI) combined with the temperature difference (ΔT1, ΔT2) between the respective drying gas (T1, T2) and the exit temperature (Ta) plays an important part.

The entry temperature T1 of the drying gas (1)-the drying gas (1) is designated 1 in FIG. 1—is the temperature of the drying gas as it is introduced into the spray cylinder (for measuring point see reference numeral 7, FIG. 1). The mass flow of the drying gas Mg represents the amount of the gas determined as mass per unit of time, while Mg1 denotes the mass flow of the drying gas (1) and Mg2 the mass flow of the drying gas (2). The exit temperature (Ta) of the drying gas may be determined, according to FIG. 1, at the measuring point 6 (reference numeral 6, FIG. 1). The entry temperature T2 of the drying gas (2)-the drying gas (2) is designated 2 in FIG. 1—represents the temperature of the drying gas (2) which can be measured before the drying gas is introduced into the spray cylinders (see reference numeral 5, FIG. 1). By the mass flow of the spray liquid (MI) (see reference numeral 3, FIG. 1) is meant the amount (determined as mass) of spray solution per unit of time. The temperature differences ΔT1 and ΔT2 in each case represent the temperature differences between the measuring points of the inventive process characterised according to FIG. 1.

According to the invention the process for preparing the inhalable powders according to the invention may be characterised by the drying coefficient V1 and the drying coefficient V2. The parameters V1 and V2 can be obtained according to the mathematical equations Equation 1 and Equation 2.

$$\begin{matrix}V 1 = \frac{Mg 1}{Ml} \cdot Δ T 1 mit Δ T 1 = T 1 - Ta & Equation 1 \\ V 2 = \frac{Mg 2}{Ml} \cdot Δ T 2 mit Δ T 2 = T 2 - Ta & Equation 2\end{matrix}$$

The process for preparing the inhalable powders according to the invention is characterised in that the entry temperature of the drying gas (1) is from 80° C. to 150° C., preferably from 90° C. to 140° C. and particularly preferably from 100 ° C. to 130° C. and the entry temperature of the drying gas (2) is between 200° C. and 400° C. Moreover, the drying of the spray mist is carried out such that the drying coefficient V1 (see Equation 1) has a value of between 100 K and 2000 K, preferably between 200 K and 1500 K and particularly preferably between 400 K and 1000 K and the drying coefficient V2 (see Equation 2) has a value of between 250 K and 4000 K, preferably between 500 K to 3000 K and particularly preferably between 1000 K and 2000 K.

Preferably, the process step of drying is characterised in that the ratio of mass flow Mg1:mass flow Mg2 is between 20:1 and 3:1.

The preparation process is also characterised in that the exit temperature of the drying gas, measured at the exit from the spray chamber, has a temperature from 40° C. to 90° C., preferably 40° C. to 90° C.

The preparation equipment shown in FIG. 1 constitutes an embodiment by way of example, by means of which the process according to the invention can be carried out. The drawing (FIG. 1) shows a modified Büchi B-191 spray dryer with secondary drying zone.

According to FIG. 1 the spray solution (3) is nebulised in the spray chamber for example using a standard commercial dual-substance nozzle. The drying gas (1) is heated and introduced into the spray chamber co-currently with the spray mist. Reference numeral (7) denotes the measuring point for the entry temperature of the spray gas (1). The drying gas (2) is heated and introduced into the spray cylinder in countercurrent. Reference numeral (5) denotes the measuring point for the entry temperature of the drying gas (2). Reference numeral (6) denotes the measuring point for the exit temperature of the drying gas, while (4) denotes the exit for the dried aerosol/drying gas.

The process according to the invention thus makes it possible to prepare inhalable powders, the particles containing a crystalline matrix forming agent, preferably mannitol. Particles according to the invention may contain active substances, the active substance or substances being incorporated in crystalline adjuvant components so that the active substance or substances are physically and chemically stabilised by this “scaffolding” of the adjuvant. In one specific embodiment it is surprisingly found that physically stable microparticles can be prepared, which permit a high proportion of active substance. Powders thus prepared are characterised by a particle size, e.g. measured by laser diffraction, by a mean particle size x₅₀ in the range from 1 μm to 10 μm, preferably from 1 μm to 6 μm. By the mean particle size in the sense used here is meant the 50% value from the volume distribution, measured with a laser diffractometer by the dry dispersion method.

According to the invention, medicament preparations are also included, which are characterised by the pre-metering of the inhalable powders into a dosage container. The dosage containers may preferably be made from a material that comprises a material selected from among the synthetic plastics, at least at the surface in contact with the inhalable powder.

Filled capsules that contain the inhalable powders according to the invention may be mentioned as examples of a preferred pre-metered medicament preparation. These capsules are filled using methods known in the art for filling the empty capsules with the inhalable powders according to the invention. It is particularly preferred to use capsules the material of which is selected from among the synthetic plastics, most preferably selected from among polyethylene, polycarbonate, polyester, polypropylene and polyethylene terephthalate. Particularly preferred synthetic plastic materials are polyethylene, polycarbonate or polyethylene terephthalate. If polyethylene is used as one of the capsule materials which is particularly preferred according to the invention, it is preferable to use polyethylene with a density of between 900 and 1000 kg/m³, preferably 940-980 kg/m³, more preferably about 960-970 kg/m³ (high density polyethylene). The synthetic plastics according to the invention may be processed in various ways using manufacturing methods known in the art. Injection moulding of the plastics is preferred according to the invention. Injection moulding without the use of mould release agents is particularly preferred. This method of production is well defined and is characterised by being particularly reproducible.

Of equivalent importance to the capsules according to the invention are powder reservoirs into which the medicament preparations according to the invention are filled so that they make contact with the product. It should be understood from this that powder reservoirs according to the invention are configured so that at least the material that makes contact with the medicament preparation is a material selected from among the synthetic plastics. In another aspect the present invention relates to the above-mentioned capsules that contain the above-mentioned inhalable powder according to the invention. These capsules may contain about 1 to 25 mg, preferably about 2 to 25 mg, particularly preferably about 3 to 20 mg of inhalable powder.

According to these embodiments, the capsules may contain 1, 2, 3, 4, 5, 6, 7, 8, 9, 10, 11, 12, 13, 14, 15, 16, 17, 18, 19, 20, 21, 22, 23, 24 or 25 mg of inhalable powder.

Moreover the present invention relates to an inhalation kit consisting of one or more of the capsules described hereinbefore, characterised in that they contain inhalable powder according to the invention, combined with a dry powder inhaler.

The present invention further relates to the use of the inhalable powders according to the invention for preparing a medicament for treating respiratory complaints, particularly for the treatment of COPD and/or asthma, characterised in that the inhaler disclosed in WO2004047796 (see FIG. 1) is used.

### EXAMPLES

### Example 1

### Drying with Additional Drying Gas (2)

Preparation of inhalable powder by spray-drying to produce embedding particles. The particles thus obtained contain a combination of active substances (glucocorticoid, anticholinergic and beta-agonist) in a crystalline mannitol matrix.

Method:

The solvent (H₂O:EtOH 1:0.9 m/m) is placed in an Erlenmeyer flask. The embedding material (mannitol) is added batchwise with vigorous stirring (magnetic stirrer) and with heating (40° C.) in the ultrasound bath. As soon as the solution is clear, the active substances are added. After they have dissolved fully, spray drying takes place immediately. The composition of the solution is given in the following Table 1.

TABLE 1
Composition of solution of Example 1.

| | |
|---|---|
| solvent (H2O:EtOH 1:0.9 m/m) | 944.6 g |
| mannitol | 13.7 g |
| ciclesonide | 6.188 g |
| beta-agonist CL | 0.059 g |
| tiotropium BR | 0.032 g |

Beta-agonist CL here denotes the substance 2H-1,4-benzoxazin-3(4H)-one, 6-hydroxy-8-[(1R)-1-hydroxy-2-[[2-(4-methoxyphenyl)-1,1-dimethylethyl]amino]ethyl]-, monohydrochloride and tiotropium BR denotes the substance tiotropium bromide, as known from European Patent Application EP 418 716 A1.

The spray-drying is carried out with a BÜCHI Mini-Spray Dryer (B-191) in conjunction with a dual substance nozzle (Büchi, 0.5 mm Art.-No. 4363). The spray dryer has been modified by removing the aspirator. N₂ is supplied through the process gas inlet as a dry gas (approx. 17 m³/h at approx. 90° C.), so that there is a flow through the apparatus in the excess pressure range (corresponding to drying gas (1)). For the second drying step ambient air is sucked in and fed into the process (approx. 3 m³/h at approx. 400° C.) (corresponding to drying gas (2)). The outlet filter between the cyclone and the aspirator has been removed and the exit gas is piped out directly. The mass flow of the nozzle gas throughput is determined using an external measuring apparatus (Kobold DSM212) and uncoupled from the original float-type flowmeter. The nozzle is operated at a gas throughput of 18 l/min (approx. 2 bar overpressure). The solvent throughput is approx. 16 g/min. The resulting exit temperature is in the region of approx. 58° C. The process parameters used are listed in Table 2.

TABLE 2
Spray drying parameters Example 1
(Drying with additional drying gas (2)).

| | |
|---|---|
| mass flow solution: MI | 0.0156 kg/min |
| spray pressure (nozzle type) | 2 bar excess pressure N₂ (BÜCHI |
| | spray nozzle 0.5 mm, modified) |
| flow volume of nozzle gas | 18 l/min (BÜCHI spray nozzle |
| (nozzle type) | 0.5 mm, modified) |
| mass flow drying gas (1): Mg1 | 0.38 kg/min |
| entry temperature drying gas (1): T1 | 90° C. |
| mass flow drying gas (2): Mg2 | 0.07 kg/min |
| entry temperature drying gas (2): T2 | 400° C. |
| exit temperature: Ta | 58° C. |
| ΔT1 | 32 K |
| ΔT2 | 342 K |
| drying coefficient: V1 | 779 K |
| drying coefficient: V2 | 1535 K |
| cross-section of drying tower | 105 mm |

The composition of the powder obtained according to Example 1 is shown in Table 3.

TABLE 3
Composition of solids particles (computed) Example 1
(Drying with additional drying gas (2)).

| solid | in 5 mg dry powder | |
|---|---|---|
| mannitol | 3.43 | mg |
| ciclesonide | 1547 | μg |
| beta-agonist CL | 14.7 | μg |
| tiotropium BR | 8.1 | μg |

Characterisation of the Resulting Particles/of the Inhalable Powder:

Characteristic properties of the inhalable powders obtained according to Example 2 are shown in Table 4. The geometric mean particle size (laser diffraction: Sympatec Dry Dispersion) was determined immediately after preparation.

Moreover, the inhalable particles were determined as the “proportion by volume<5 μm after delivery”. By this is meant the amount of powder comprising particles smaller than 5 μm (given in % percent by volume, measured by laser diffraction. The aerosol mist is produced by fragmentation of the sample by expulsion from an inhaler (Handihaler)—for more details see the section on Methods.

It is clear that the “proportion by volume<5 μm after delivery” of the inhalable powders according to Example 1 is stable. The reduction in the “proportion by volume<5 μm after delivery”after storage (1 week, open, 40° C./75% r.h.) is negligible. A reduction of less than 5 percentage points, preferably less than 4 percentage points, more particularly less than 3 percentage points, and most particularly preferably less than 2 percentage points, and most exceptionally preferably less than 1 percentage point after storage (1 week, open, 40° C./75% r.h.), is to be regarded as negligible. By the term percentage points is meant a percentage based on 100% (percent by volume).

TABLE 4
Powder properties of Example 1 (Drying with additional drying gas (2)).
Symatec Dry Dispersion

| | |
|---|---|
| particle size x₅₀ | 2.9 μm |

“Proportion by volume <5 μm after delivery” from
an inhaler (HandiHaler):

| | directly after preparation/ | 1 wk |
|---|---|---|
| flow rate | spray drying | 40° C./75% |
| 39 l/min: | 69% | 69% |
| 60 l/min: | 76% | 76% |

### Methods of Measurement

I) Determining Particle Size by Laser Diffraction (Sympatec Dry Dispersion) in Order to Determine the Average Particle Size x₅₀:

Measuring Equipment and Settings:

The equipment was operated according to the manufacturers instructions.
Measuring equipment: Laser diffraction spectrometer (HELOS), Sympatec (particle size determined by Fraunhofer diffraction)
Dispersing unit: RODOS dry disperser with suction funnel, Sympatec
Sample quantity: 200 mg±150 mg
Product feed: Vibri Vibrating channel, Messrs. Sympatec
Frequency of vibrating channel: rising to 100
Duration of sample feed: 15 to 25 sec. (in the case of 200 mg)
Focal length: 100 mm (measuring range: 0.9-175 μm)
Measuring time/waiting time: about 15 s (in the case of 200 mg)
Cycle time: 20 ms
Start/stop at: 1% on channel 28
Dispersing gas: compressed air
Pressure: 3 bar
Vacuum: maximum
Evaluation method: HRLD

Sample Preparation/Product Feed:

About 200 mg of the test substance are weighed onto a piece of card.

Using another piece of card all the larger lumps are broken up. The powder is then sprinkled finely over the front half of the vibrating channel (starting about 1 cm from the front edge).

After the start of the measurement the frequency of the vibrating channel is varied so that the sample is fed in as continuously as possible. However, the amount of product should not be so great that adequate dispersion cannot be achieved.

II) Determining the “Proportion by Volume<5 μm After Delivery “as the Amount Delivered from an Inhaler (HandiHaler):

The HandiHaler® inhaler is used for the measurement. The inhalable powder to be analysed is packed into size 3 plastic capsules (polyethylene) as disclosed in EP 1100474. The inhalation capsules are filled with 20 mg.

Method:

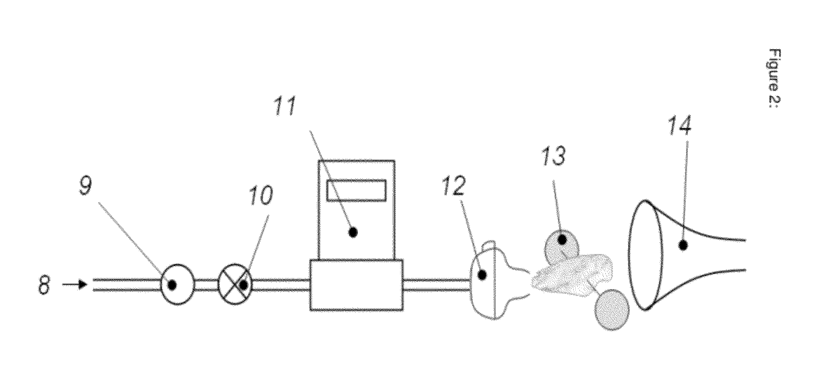

(The delivery to determine the “proportion by volume<5 μm after delivery” is carried out using the technical set-up as shown in FIG. 2.)

The HandiHaler® is operated by compressed air (8) via a gas connection to the inlet opening of the capsule chamber. The flow rates used are 39 l/min and 60 l/min (preferably 39 l/min, as this corresponds to a drop in pressure at the HandiHaler® of 4 kPa). Using a time-controlled 2-way magnetic valve (9) compressed air is supplied to the inhaler (12) for a period of 10 seconds. The flow rate is adjusted using a throughflow control valve (10) and the flow rate is monitored using a Kobold DMS-614C3FD23L mass flow flowmeter (11).

The particle size distribution is determined directly on the aerosol mist, by measuring the particle size at a distance of 2±0.5 cm behind the powder exit from the inhaler using a HELOS laser diffractometer made by Sympatec GmbH, Clausthal-Zellerfeld (13). Directly behind the measuring zone the particles are sucked up with a vacuum cleaner (14).

Measuring Conditions:

The focal width of the laser diffractometry is f=100 mm (measuring range: 0.9-175 μm). The evaluation is carried out in high resolution mode (Fraunhofer HRLD, software version WINDOX 4.1.2.0) assuming a spherical model (form factor=1).

Evaluation:

The target value “proportion by volume<5 μm after delivery” corresponds to the proportion by volume of the particles, given in percent, that are smaller than 5 μm.

III) Determining the Crystallinity

Measuring Equipment and Settings:
Measuring equipment: temperature modulated DSC (TMDSC) Q1000 TA Instruments
Test crucible: standard crucible, perforated
Sample quantity: 10 mg±2 mg
Modulation: ±0.54° C., period 40 seconds
Heating rate: 5° C./min
Temperature range: −40° C. to 200° C.

Evaluation:
Software: TA Instruments Universal 2000 (Version 4.2)
RevCP-Signal: Smooth=4; stage analysis Tg
1. Tg [° C.]: middle point of the Cp step from the RevCp signal

2. ΔCp [J/(g·° C.)]: increase in the heat capacity on glass transition from the RevCp signal

The glass stage is determined using TA Instruments Software (Universal 2000, Version 4.2) via the “Analyze/Glass Transition . . . ” function, from the RevCP signal. The cut-off points are applied to the baseline before and after the glass stage as described for example by McPhillips et al. [McPhillips, H.; Craig, D. Q. M.; Royall, P. G.; Hill, L.: Characterisation of the glass transition of HPMC using modulated temperature differential scanning calorimetry; International Journal of Pharmaceutics (1999) No. 180, 83-90].

If the sample contains amorphous fractions, a Cp increase may be observed at the glass transition of the sample (ΔCp_{(P)}). For a sample of this kind, the degree of crystallinity may be determined from the variables of the Cp increase at the glass transition of the sample (ΔCp_{(P)}), the Cp increase of the totally amorphous matrix forming agent (ΔCp_{(M,a)}) and the proportion of the matrix forming agent (A_{(M)}) present in the sample, according to Equation 3,

$$Kristallinität [ \% ] = 100 - \frac{Δ {Cp}_{( P )} \cdot 10000}{Δ {Cp}_{( M , a )} \cdot {A}_{( M )}} \qquad ( Equation 3 )$$

wherein
ΔCp_{(P)}[J/(g·° C.)]: denotes the Cp increase at the glass transition of the sample
ΔCp_{(M,a)}[J/(g·° C.)]: denotes the Cp increase at the glass transition of the totally amorphous matrix forming agent
A_{(M)}[%]: denotes the proportion by mass of the matrix forming agent in the sample.

Amorphous Reference Material for Determining the Crystallinity:

For determining ΔCp_{(M,a)} according to Equation 3 the matrix forming agent is required in amorphous form as reference material. The amorphous reference substance is prepared for example by melting and rapidly cooling (quenching) the substance. To do this, 10±2 mg of the matrix forming agent is weighed into a DSC crucible and heated to about 10 to 30° C. above the melting temperature in the TMDSC apparatus. The crucible is removed at this temperature and immediately plunged into deep-frozen liquid nitrogen. The Cp increase ΔCp_{(M,a)} is determined by placing the sample of the totally amorphous matrix forming agent, once it has been prepared, in the oven of the TMDSC apparatus and measuring it. Measurement is started at least 20° C. below the expected glass transition point. Measurement is carried out according to the equipment parameters listed above (TA Instruments Software; Universal 2000, Version 4.2) via the “Analyze/Glass Transition . . . ”) function.

IV) Determining the Droplet Size by Laser Diffraction
Measuring method: In order to determine the droplet size the spray cone (spray) of the nozzle is analysed directly in the laser measuring zone in terms of the droplet size distribution. By the median value X₅₀ is meant the droplet size below which 50% of the droplets lie. The characteristic value Q_{(5.8)} describes the percentage fraction of droplets that are less than 5.8 μm in size. H₂O is used as the solution. The characteristic value is referred to as the average droplet size X₅₀.
Measuring device: Laser diffraction spectrometer (HELOS), Messrs. Sympatec
Software: WINDOX Version 4
Dispersing unit: RODOS/dispersing pressure: 3 bar
Focal width: 100 mm [measuring range: 0.9 . . . 175 μm]
Evaluation mode: Mie (V 4)

1. A process for preparing inhalable powders containing a crystalline matrix forming agent, which is selected from among sugars, polyols, polymers, amino acids, proteins, di-, tri-, oligo- and polypeptides; as well as one or more pharmaceutical active substances,
characterised in that a spray solution that comprises the matrix forming agent and the pharmaceutical active substance is spray-dried, comprising the steps of:
(a) dissolving one or more active substances and the matrix forming agent in water, an organic solvent or an organic-aqueous solvent mixture in order to prepare a solution with a dissolved solids content of between 1% by weight and 20% by weight,
(b) spraying the solution thus obtained in the conventional manner, so as to obtain a spray mist with a droplet size having
(i) the characteristic value Q_{(5.8)} of between 50% and 100% and
(ii) the average droplet size X₅₀ in the range from 1 μm to 20 μm,
(c) drying the resulting spray mist using a drying gas while applying the following parameters:
(i) an entry temperature of the drying gas (1) of from 80° C. to 200° C., and
(ii) subjecting the aerosol to secondary drying in the spray chamber using a second drying gas (2), the temperature of the drying gas (2) being between 200° C. and 400° C.,
(iii) the ratio of the volume flow of drying gas (1) : drying gas (2) being between 20:1 and 3:1,
(iv) the drying gas coefficient V1 being between 100 K and 2000 K and the drying coefficient V2 being between 250 K and 4000 K and
(v) an exit temperature of the drying gas of from 40° C. to 90° C. and
(d) separating the dried particles of solid from the current of drying gas in conventional manner.

2. The process according to claim 1 for preparing inhalable powders containing a crystalline matrix forming agent, characterised in that the matrix forming agent is selected from among a polyol.

3. The process according to claim 2 for preparing inhalable powders containing a crystalline matrix forming agent, characterised in that the matrix forming agent is mannitol.

4. The process according to claim 1, characterised in that the active substance or substances is or are selected from among anticholinergics, betamimetics, steroids, phosphodiesterase-IV-inhibitors, LTD4-antagonists, EGFR-kinase inhibitors, dopamine agonists, H1-antihistamines, PAF-antagonists, P13-kinase inhibitors, P38 MAP-kinase inhibitors, antiallergics and phosphodiesterase-V-inhibitors.

5. The process according to claim 1, characterised in that the temperature of the drying gas (2) is between 300° C. and 380° C.

6. The process according to claim 1 characterised in that the ratio of the volume flow of drying gas (1):drying gas (2) is between 18:1 and 10:1 (ratios by mass).

7. The inhalable powder obtained by the process according to claim 1, characterised in that it contains mannitol as matrix forming agent and an EGFR-inhibitor as active substance, the ratio of active substance:matrix forming agent being between 1:1 to 3:1 (ratios by mass).

8. The inhalable powder obtained by the process according to claim 1, characterised in that it contains mannitol as matrix forming agent and a combination of an anticholinergic, betamimetic and steroid as active substance, the ratio of the total of active substances:matrix forming agent being between 1:1 to 3:1 (ratios by mass).

9. An inhalation kit consisting of an inhalation device that can be used to administer inhalable powders from powder-containing capsules, and an inhalable powder according to claim 7.

10. An inhalation kit consisting of an inhalation device that can be used to administer inhalable powders from powder-containing capsules, and an inhalable powder according to claim 8.

11. The process according to claim 1, wherein the dissolved solids content in step (a) is between 2% by weight and 10% by weight.

12. The process according to claim 1, wherein the dissolved solids content in step (a) is between 3% by weight and 8% by weight.

13. The process according to claim 1, wherein the average droplet size X₅₀ in step b(ii) is in the range from 1 μm to 8 μm.

14. The process according to claim 1, wherein the average droplet size X₅₀ in step b(ii) is in the range from 1 μm to 3 μm.

15. The process according to claim 1, wherein the entry temperature of the drying gas (1) in step (c) (i) is from 90° C. to 160° C.

16. The process according to claim 1, wherein the entry temperature of the drying gas (1) in step (c) (i) is from 100° C. to 150° C.
